(19) United States
(12) Patent Application Publication
(10) Pub. No.: US 2012/0271230 A1
(43) Pub. Date: Oct. 25, 2012

(54) Fibroid Treatment System and Method

(75) Inventors: Kevin R. Arnal, Excelsior, MN (US); Thomas Q. Dinh, Minnetonka, MN (US); Edouard A. Koullick, Golden Valley, MN (US); Rosa K. Canifax, Bloomington, MN (US)

(73) Assignee: AMS Research Corporation, Minnetonka, MN (US)

(21) Appl. No.: 13/403,781

(22) Filed: Feb. 23, 2012

Related U.S. Application Data
(60) Provisional application No. 61/445,802, filed on Feb. 23, 2011.

(51) Int. Cl. A61B 17/42; A61M 5/00

(52) U.S. Cl. 604/93.01; 606/119

(57) ABSTRACT

Various embodiments of a fibroid treatment system and method are provided. Embodiments are adapted to treat fibroids utilizing by blocking or impeding blood flow through the uterine artery to prevent growth of the fibroid. Additionally, drugs or therapeutic agents can be included to further shrink or starve off the fibroid to provide a secondary mode of action.

### PRIORITY

This Application claims priority to and the benefit of U.S. Provisional Patent Application No. 61/445,802, filed Feb. 23, 2011 and entitled “Fibroid Treatment System and Method,” which is incorporated herein by reference in its entirety.

### FIELD OF THE INVENTION

The present invention relates generally to surgical methods and apparatus and, more specifically, to systems and methods for treating fibroids, e.g., uterine fibroids.

### BACKGROUND OF THE INVENTION

Uterine fibroids are noncancerous growths of the uterus that are common in women and are not associated with an increased risk of uterine cancer and almost never develop into cancer. Uterine fibroids, also called fibromyomas, leiomyomas or myomas, develop from the smooth muscular tissue of the uterus (myometrium). A single cell reproduces repeatedly, eventually creating a pale, firm, rubbery tissue mass distinct from surrounding or neighboring normal myometrium. Fibroids range in size from seedlings, undetectable by the human eye, to bulky masses that can distort and enlarge the uterus. They can be single or multiple, in extreme cases expanding the uterus so much that it reaches the rib cage.

Uterine fibroids are clinically apparent in 20% to 25% of women during the reproductive years and cause symptoms necessitating treatment, typically surgical removal of the uterus. Such signs and symptoms are influenced by fibroid size and location and include heavy or prolonged menstrual bleeding, pelvic pressure or pain, urinary incontinence, frequent urination or urine retention. Fibroids that grow into the inner cavity of the uterus (submucosal fibroids) are believed primarily responsible for prolonged, heavy menstrual bleeding. Fibroids that project to the outside of the uterus (subserosal fibroids) can press on the bladder or ureters, causing urinary symptoms. Subserosal fibroids that bulge from the posterior of the uterus can press either on the rectum, causing constipation, or on spinal nerves, causing backache. A fibroid that hangs by a stalk inside or outside the uterus (pedunculated fibroid) can trigger pain by turning on its stalk. Fibroids that are intermediate the inner cavity and the outer surface of the uterus (intramural fibroids) may also be symptomatic.

Uterine fibroids are highly vascularized and encapsulated such that an avascular space separates the distinct outer encapsulating surface of the fibroid from normal myometrium. The normal myometrium is also well vascularized, and a more limited number of a blood vessels cross the avascular space to nourish the fibroid. Thus, it has been observed that a uterine fibroid can be readily excised from the avascular space after the uterus is surgically exposed in a myomectomy procedure. However, the surgical exposure of the uterus through the abdominal wall in a myomectomy procedure can involve risks and complications, and fibroids may recur over time ultimately necessitating a repeated myomectomy or ultimately a hysterectomy.

Consequently, less invasive procedures have been proposed to shrink or remove fibroids. Fast growing fibroids require more oxygenated blood than normal myometrium. Consequently fibroid growth can be halted and reversed by blocking uterine artery blood flow in a minimally invasive uterine artery embolization procedure, which reduces the oxygenated blood supply to the uterus. This procedure involves some risk of ischemia of the normal myometrium and ovaries.

Notwithstanding these advances, a need remains for a simple, minimally invasive instrument and procedure for accessing and removing a uterine fibroid from the avascular space.

### SUMMARY OF THE INVENTION

The present invention describes various systems and methods adapted to treat uterine fibroids in a patient. Embodiments of the present invention are adapted to treat fibroids utilizing mechanical means to block, restrict or otherwise impede blood flow through a uterine artery to prevent growth of the fibroid. Additionally, drugs or therapeutic agents can be included to further shrink, block or starve off the fibroid.

Certain embodiments can include one or more clip devices adapted to engage or shroud at least a portion of the patient's uterine artery to completely or partially cut off blood through the artery.

Other embodiments can include a bulking agent or device adapted to cut off, completely or partially, the uterine artery to cut off the blood flow to the target fibroid. The bulking agent is adapted and placed to assert pressure on the outside diameter of the uterine artery.

Still other embodiments can include one or more occlusion members that are introduced into and along a portion of the uterine artery to block off, at least partially, blood flow through the artery and to the corresponding fibroid.

### BRIEF DESCRIPTION OF THE DRAWINGS

FIGS. 1-2 show female anatomical structures and features relevant for the uterine fibroid treatment systems and methods provided in accordance with embodiments of the present invention.

FIGS. 3-4A show clamp or like devices adapted to restrict or cut off blood flow through a uterine artery in accordance with embodiments of the present invention.

FIGS. 5-6 show an introduction tool for introducing or deploying a device adapted to restrict or cut off blood flow through a uterine artery in accordance with embodiments of the present invention.

FIG. 7 shows a bulking agent or member adapted to restrict or cut off blood flow through a uterine artery in accordance with embodiments of the present invention.

FIG. 8 shows occlusion members within uterine arteries to restrict or cut off blood flow through the artery in accordance with embodiments of the present invention.

FIGS. 9-10 show occlusion members in accordance with embodiments of the present invention.

### DETAILED DESCRIPTION OF PREFERRED EMBODIMENTS

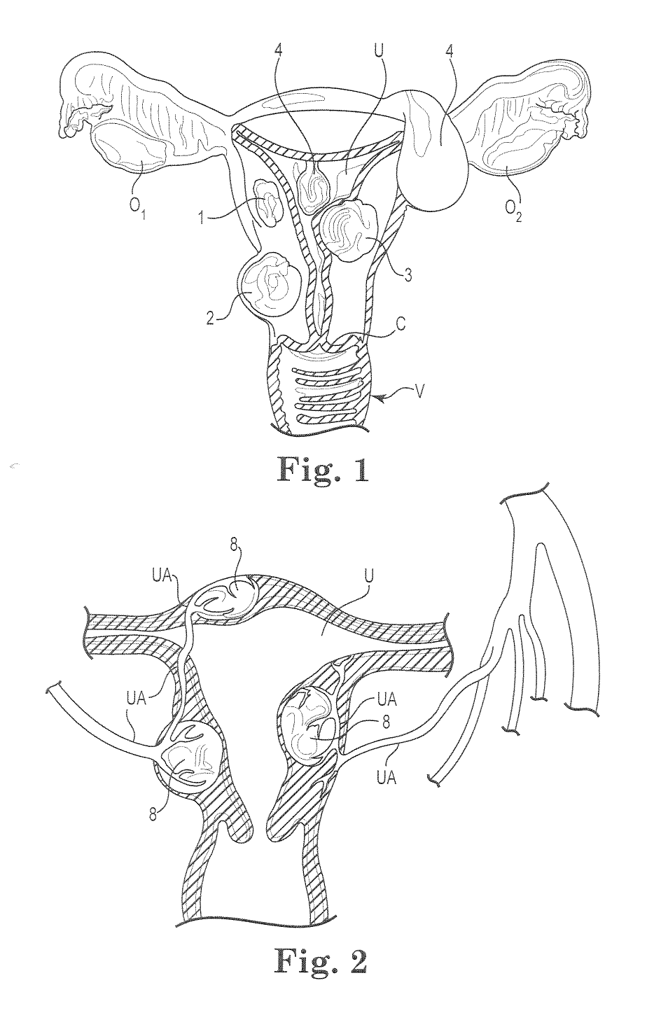

A variety of uterine fibroids are illustrated in FIG. 1 in relation to the female uterus U, ovaries O1 and O2, the vagina V and the cervix C. The uterus U has a pear-shaped, uterine body extending between a fundus extending right and left to junctions with the right and left Fallopian tubes joined with ovaries O1, O2 and to the cervix C that extends to the vagina. The uterus U is formed of a smooth muscle uterine wall or myometrium bounded by an outer surosa membrane and an interior uterine mucosa (endometrium) that lines the uterine cavity.

There are several types of uterine fibroids, some of which are illustrated in FIG. 1, including an intramural uterine fibroid 1, a subserosal uterine fibroid 2, and a submucosal uterine fibroid 3 generally within the myometrium, and pedunculated uterine fibroids 4 extending on pedestals from the myometrium. The subserosal uterine fibroid 2 is located just under the uterine serosa and may be attached to the corpus by a narrow or a broad base. The intramural uterine fibroid 1 is within the thick myometrium and may distort the uterine cavity or cause an irregular external uterine contour. The submucosal uterine fibroid 3 is located within the myometrium proximate the endometrium such that it bulges the endometrium into the uterine cavity.

As shown in FIG. 2, discrete blood vessels, such as the uterine artery UA and vessels extending therefrom, nourish the cells of the fibroid masses 1-4. These uterine fibroids are generally referred to individually herein as a fibroid mass 8. The fibroid mass 8 is generally a well circumscribed, solid, and typically benign fibroid masses or tumors composed of smooth muscle cells and extracellular matrix.

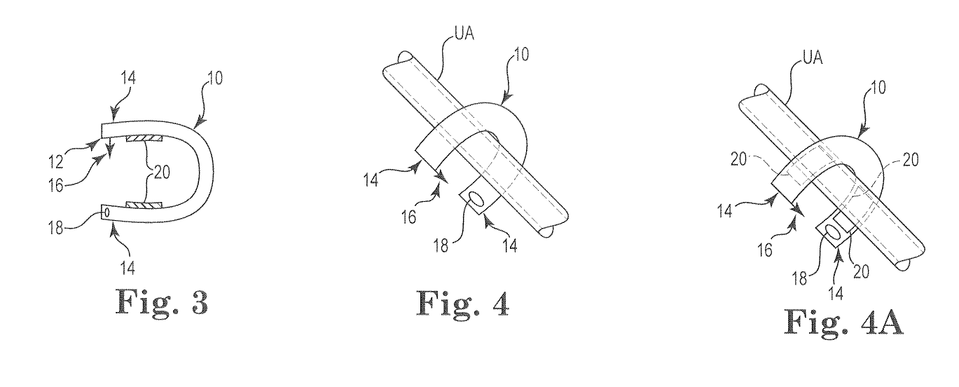

Referring generally to FIGS. 3-10, various embodiments of devices and methods are provided to treat fibroids or fibroid masses 8 (e.g., uterine fibroids) by cutting off or reducing blood through, or otherwise directing treatment to the uterine artery UA of the patient. Various portions of the devices or systems can be constructed of metal or polymer materials, such as Nitinol™, polypropylene, polyethylene, fluoropolymers or like compatible materials.

The various teachings, devices and methods disclosed in U.S. Patent Publication No. 2007/0225745 can be used in conjunction with the various embodiments disclosed herein and is, thereby, incorporated herein by reference in its entirety.

As shown in FIGS. 3-4A, the present invention can include one or more clip devices 10 adapted to engage or surround at least a portion of the patient's uterine artery UA to completely or partially cut off blood through the artery. The devices 10 are adapted and placed to assert pressure (e.g., squeeze) on the outside diameter of the uterine artery UA to reduce or cut off the body lumen and blood flow therethrough. Cutting off or reducing blood flow through the artery will, in turn, cut off blood to the corresponding fibroid mass 8, thereby causing the fibroid 8 to shrink or disappear over time. As depicted, the device 10 can be generally C-Shaped or U-Shaped, and it can be made of a myriad of known materials. Other shapes or constructs are envisioned as well, such as semi-circular or completely circular. Such devices 10 can vary in shape as well to accommodate the outer diameter of the corresponding target uterine artery UA location. The device 10 can be generally flexible or malleable in various embodiments to accommodate deployment and shrouding engagement with the uterine artery UA.

In certain embodiments, the device 10 can be constructed of a shape memory material (e.g., polymer or metal) allowing for expandability during deployment and constriction bias upon placement around the respective portion of the uterine artery UA. Further, embodiments of the device 10 can include one or more fastener or locking mechanisms 12 proximate one or more free ends 14 of the device 10. The mechanism 12 can include a protruding member 16 adapted to selectively or lockingly engage with a corresponding feature 18 (e.g., aperture, member, mechanism or device) provided with the opposing free end 14. Other known clips, fasteners, adhesives, and like engagement devices, features and techniques known to those of ordinary skill in the art can be employed without deviating from the spirit and scope of the present invention. In addition, the device 10 can take on the form of a tie-off device, lapro restriction device or other known surgical devices and techniques adapted to restrict or constrict body lumens.

In various embodiments, the device 10 will not completely cut off or restrict all blood flow through the respective uterine artery UA. Such partial-obstruction embodiments can be implemented to prevent or reduce pain, cramping and like discomforts that may occur if the blood flow is completely blocked through the uterine artery UA.

Further, as shown in FIGS. 3 and 4A, the device 10 can include a drug, agent or treatment portion or coating 20 to facilitate obstruction, tissue treatment, and the like, at the targeted portion of the uterine artery UA. The treatment portion 20 can be included along any surface of the device 10. In certain embodiments, the treatment portion 20 can be included along a portion of the device 10 within the interior C-shaped construct that is designated for contact with the outer diameter of the uterine artery UA.

Exemplary drugs or agents included with the treatment portion or coating 20 can include drugs that inhibit the growth of fibroids to prevent future recurrence. Such drugs can include antiproliferative drugs such as Rapamycin (Sirolimus), doxorubicin, Taxol and the like. Other anti-inflammatory and pain relief drugs can be used as well, including NSAIDs, lidocaine, ropivacaine, steroids, etc. Polymers such as PLGA and other biostable polymers like polyurethanes and silicone can be used as a carrier for the drugs mentioned herein.

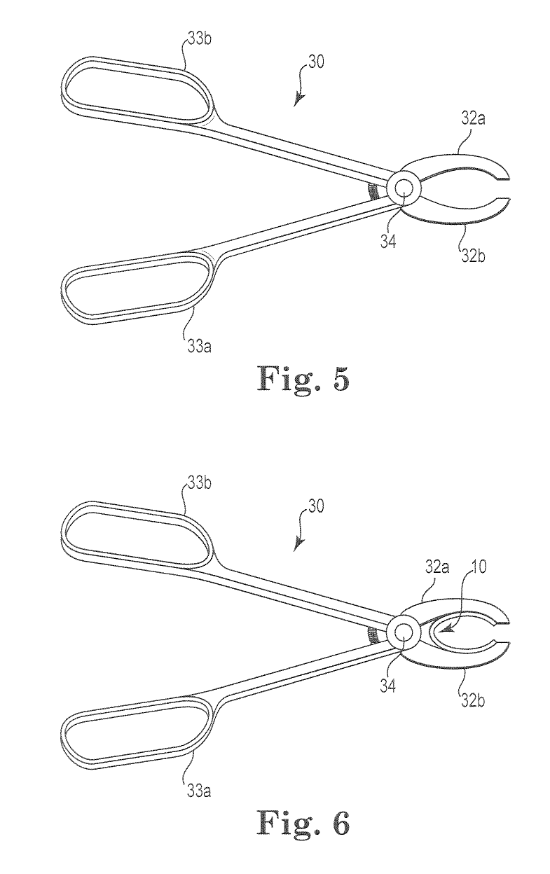

FIGS. 5-6 shows an exemplary delivery and deployment tool 30 for use with the device 10. Namely the tool 30 is adapted to introduce the device 10 and deploy the device 10 to the target uterine artery UA location. In such embodiments, the tool 30 can include one or more jaw portions 32a, 32b pivotally connected at portion 34 with movement of corresponding handle portions 33a, 33b. The jaws 32a, 32b are generally constructed and shaped to receive the device 10 therein. Upon positioning of the device 10 around the target uterine artery UA location, the jaws 32a, 32b can be clamped or moved a distance closer to one another such that the C-shaped device is engaged around the uterine artery UA. As disclosed herein, the device 10 can include the locking or securement mechanism 12 that can also be engaged during this clamping operation of the tool 30 to retain the device 10 securely around the uterine artery UA. Other tools, device and techniques for introducing and deploying a clamp or like C-shaped device can be employed as well without deviating from the spirit and scope of the present invention, including clamping devices, forceps, endoscopic tools, laproscopic tools, cannula or catheter introducers, and the like. Doppler, ultrasound, x-ray and other like technologies, devices and systems can be employed with the tool 30 to facilitate navigation of the tool 30 and device 10 during introduction and deployment to the uterine artery UA.

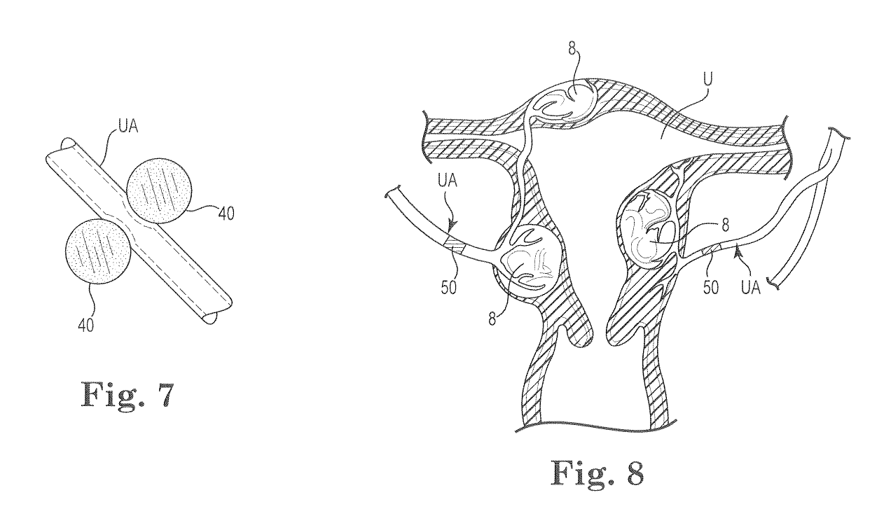

FIG. 7 depicts a bulking agent, device or substance 40 adapted to cut off, completely or partially, the uterine artery UA providing blood flow to the target fibroid 8. The bulking agent 40 is adapted and placed to assert pressure on the inside diameter or lumen of the uterine artery UA to reduce or cut off the body lumen and blood flow therethrough. The bulking agent 40 can be injected into the tissue of the uterine artery UA, or the tissue around the uterine artery UA, for applying pressure to the lumen (e.g., reduce lumen) to restrict blood flow. The bulking agent 40 can be constructed of various materials, including but not limited to collagen or like materials. Again, this procedure can limit tissue erosion and minimize inflammation and inflow response. Further, materials, tools, devices and techniques disclosed in U.S. Patent Application Publication Nos. 2011/0008299, 2009/0287239, and 2008/0171905 are envisioned for use, in whole or in part, with the present invention and are, therefore, incorporated by reference herein in their entirety.

In various embodiments, the bulking agents embodiments can include injecting a microsphere mixture of PLGA, carbon microbeads, collagen paste, alginate gel, and the like (e.g., that gels in situ). In these bulking agent mixtures, drugs can also be incorporated to help with treating fibroids growth and pain as disclosed herein.

FIG. 8 shows one or more occlusion members 50 that can be introduced into and along a portion of the uterine artery UA to block off, at least partially, blood flow through the artery and to the corresponding fibroid 8. The occlusion member 50 can be constructed of various materials, including biodegradable and non-biodegradable materials. In certain embodiments, the occlusion member 50 is a collagen material or microsphere injected or otherwise provided in the artery to block blood flow. Exemplary microspheres can include those fabricated from biodegradable polymers like PLGA, PVA (polyvinyl alcohol) or biopolymers such as alginate and chitosan microspheres. Other gels such as poly-N-acetyl glucosamine can be used as well. These gels and microspheres can allow the blocking of blood flow from a few hours to a few days. To reduce pain caused by blood flow obstruction, medications such as analgesics pain drugs like lidocaine, NSAIDs and anti-inflammatory drugs can be incorporated into these gels or microspheres to provide pain relief. The gels or microspheres can be injected into the uterine artery with a catheter or other like delivery device adapted to traverse through the artery to the target occlusion location.

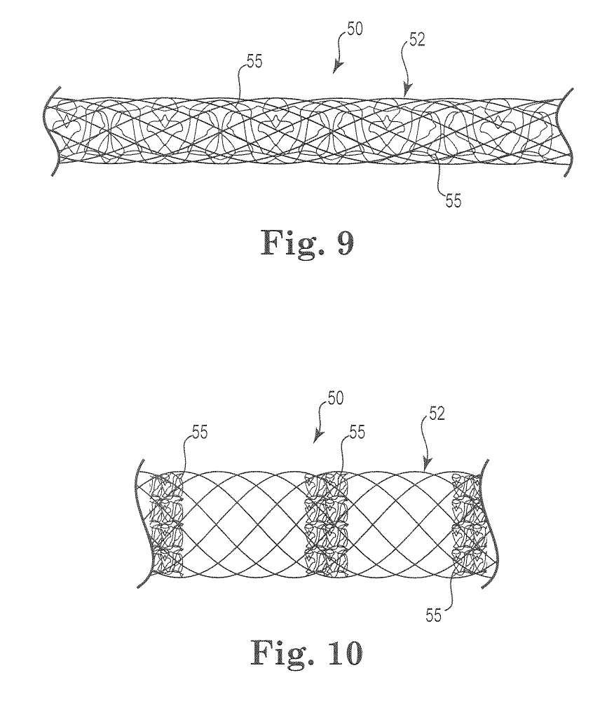

Other embodiments of the member 50 can include an occluding stent device 52, as shown in FIGS. 9-10. For those embodiments including an occluding stent 52, the stent 52 can be adapted and deployed so that only a portion is completely expanded to compress against the artery wall. The other portion can be partially expanded to obstruct blood flow. The stent 52 can include bulking agents therein, or a plurality of materials or substances 55 adapted to provide tissue ingrowth and/or decrease the open spaces within the lumen of the stent 52 to promote occlusion effects. In certain embodiments, the stent 52 is constructed of a mesh-like material, and could include polymer or metal materials.

Various embodiments of the present invention can implement other devices and techniques to restrict or occlude a portion of the target uterine artery UA to restrict or completely cut off blood flow therethrough. For instance, morcellation, such as laser morcellation can be employed.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the teachings herein. It is therefore to be understood that within the scope of the appended claims, the invention may be practiced other than as specifically described herein.

1. A uterine fibroid treatment system, comprising:
a generally C-shaped clamp device having;
first and second free ends;
an interior surface; and
wherein the generally C-shaped device is adapted to surround at least a portion of a uterine artery such that the interior surface is in contact with the uterine artery to at least partially block the flow of blood to a uterine fibroid.

2. The system of claim 1, wherein at least one of the first and second free ends includes a locking member to retain the generally C-shaped device securely around the uterine artery.

3. The system of claim 1, wherein the interior surface of the generally C-shaped device includes a treatment coating.

4. The system of claim 3, wherein the treatment coating includes a drug.

5. The system of claim 4, wherein the drug includes an antiproliferative drug.

6. The system of claim 1, wherein the generally C-shaped device is constructed at least in part of a shape-memory material.

7. The system of claim 1, further including an introducer tool adapted to introduce and deploy the generally C-shaped device.

8. The system of claim 7, wherein the introducer tool includes first and second pivoting jaws adapted to at least partially receive the generally C-shaped device.

9. A uterine fibroid treatment system, comprising:
a generally C-shaped clamp device having;
first and second free ends;
an interior surface;
an introducer tool having first and second jaw portions pivotally connected at a pivot portion, with at least one of the first and second jaw portions adapted to receive the generally C-shaped device; and
wherein the generally C-shaped device is adapted to surround at least a portion of a uterine artery upon deployment from the introducer tool such that the interior surface of the generally C-shaped device is in contact with the uterine artery to at least partially block the flow of blood to a uterine fibroid.

10. The system of claim 9, wherein at least one of the first and second free ends includes a locking member to retain the generally C-shaped device securely around the uterine artery.

11. The system of claim 9, wherein the interior surface of the generally C-shaped device includes a treatment coating.

12. The system of claim 11, wherein the treatment coating includes a drug.

13. The system of claim 12, wherein the drug includes an antiproliferative drug.

14. The system of claim 9, wherein the generally C-shaped device is constructed at least in part of a shape-memory material.

15. A method of treating uterine fibroids, comprising:
providing an occlusion member;
introducing the occlusion member into a portion of a uterine artery; and
positioning the occlusion member in the uterine artery to at least impede the flow of blood flow through the uterine artery to a uterine fibroid.

16. The method of claim 15, wherein providing an occlusion member includes providing a stent-like device, with at least a portion of the stent-like device adapted to press against an interior wall portion of the uterine artery.

17. The method of claim 16, wherein the stent-like device is constructed of a mesh material having a plurality of pores.

18. The method of claim 17, wherein the stent-like device further includes a plurality of members configured to reduce empty space within the stent-like device.

19. The method of claim 16, wherein the stent-like device includes a bulking agent.

20. The method of claim 15, wherein the occlusion member is constructed of a mesh material.
